(19) United States
(12) Patent Application Publication
(10) Pub. No.: US 2009/0171104 A1
(43) Pub. Date: Jul. 2, 2009

(54) PROCESS FOR THE PREPARATION OF ORLISTAT

(76) Inventors: Killol Patel, Junagadh (IN); Seema Kanwar, Chandigarh (IN); Keshav Deo, Gurgaon (IN); Mohan Prasad, Gurgaon (IN)

Correspondence Address: RANBAXY INC., 600 COLLEGE ROAD EAST, SUITE 2100, PRINCETON, NJ, 08540, US

(21) Appl. No.: 12/089,245

(22) PCT Filed: May 10, 2006
(86) PCT No.: PCT/IB2006/002781

§ 371 Date: Nov. 6, 2008

(30) Foreign Application Priority Data
Oct. 5, 2005 (IN) 2669/DEL/2005

(51) Int. Cl. C07D 305/12

(52) U.S. Cl. 549/328

(57) ABSTRACT

The present invention provides a process for preparing orlistat (I) by alkanoylating an amino orlistat using formic acid anhydride as an alkanoylating agent to obtain orlistat substantially free of the byproduct, (S)-N-acetylleucine (1S)-1-[[(2S, 3S)-3-hexyl-4-oxo-2-oxetanyl]methyl] dodecyl ester (deformyl-N-acetyl orlistat).

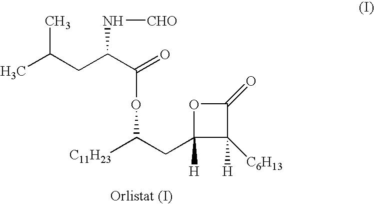

### FIELD OF THE INVENTION

The present invention relates to an improved process for the preparation of orlistat, N-formyl-L-leucine derivative, in high purity.

### BACKGROUND OF THE INVENTION

Orlistat, a tetrahydrolipstatin, is a useful pancreatic lipase-inhibiting agent and can be used for the prevention and treatment of obesity and hyperlipaemia. Chemically, orlistat is (S)-N-formyl leucine (S)-1-[[(2S, 3S)-3-hexyl-4-oxo-2 oxetanyl]methyl] dodecyl ester (orlistat) and is known from U.S. Pat. No. 4,598, 089. It is represented by Formula I,

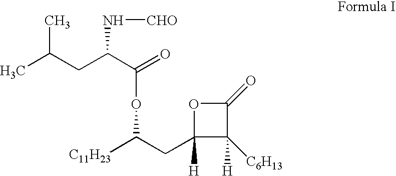

Several processes have been reported for the preparation of orlistat, such as in U.S. Pat. No. 4,202,824; U.S. Pat. No. 4,983,746; U.S. Pat. No. 4,931,463; J. Org. Chem. 1988, 53, 1218-1221; Tetrahedron Lett. 1990, 31, 3645-3648; Synlett, 1991, 11, 781-782; J. Org. Chem. 1991, 56, 4714-4718; J. Org. Chem. 1993, 58, 7768-7781; and J. Chem. Soc, Perkin Trans. 1, 1998, 17, 2679-2686.

U.S. Pat. No. 4,931,463 and WO 05/005403 disclose the use of formic acid/acetic acid anhydride for alkanoylating amino orlistat of Formula II,

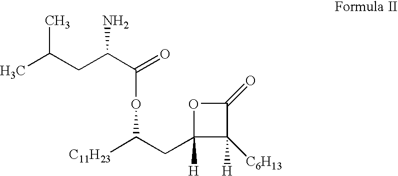

to produce orlistat.

U.S. Pat. No. 4,983,746 and WO 05/005403 disclose the use of formic acid anhydride, acetic acid anhydride or a mixed acid anhydride, such as formic acid/acetic acid anhydride, for alkanoylating amino orlistat. However, only formic acid/acetic acid anhydride is exemplified (Example 2).

The prior art mentioned above discloses the use of formic acid anhydride as an alkanoylating agent but does not contain any teaching about the effect of formic acid anhydride as the alkanoylating agent on the preparation of orlistat of Formula I. It has been observed that there are certain disadvantages in using acetic anhydride and mixed anhydride of formic acid/acetic acid anhydride as an alkanoylating agent. In particular, a byproduct of this reaction is a compound of Formula III,

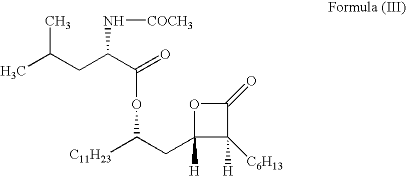

which is chemically (S)-N-acetylleucine (1S)-1-[[(2S, 3S)-3-hexyl-4-oxo-2-oxetanyl]methyl] dodecyl ester (deformyl-N-acetyl orlistat).

Therefore, there has been an ongoing search for new alkanoylating agents, which are capable of introducing the formyl group with a good yield without producing byproducts. As a result of this endeavor to find a simple, efficient, cost-effective process for the manufacture of orlistat in high yield and purity, the present inventors have surprisingly found that the selection of the alkanoylating agent during alkanoylation of amino orlistat influences the amount of byproducts formed. In the prior art methods, the use of the formic acid/acetic acid anhydride used as alkanoylating agent leads to the formation of byproduct of Formula III.

### SUMMARY OF THE INVENTION

In one general aspect, the present invention provides a process for preparing orlistat of Formula I, the process comprising

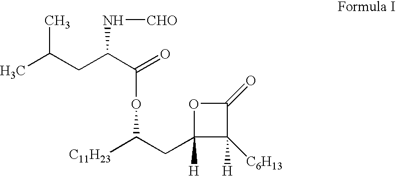

alkanoylating an amino orlistat of Formula II

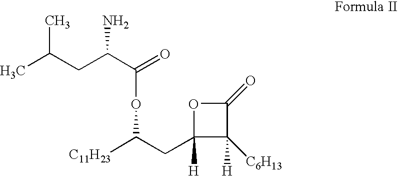

using formic acid anhydride as an alkanoylating agent to obtain orlistat, substantially free of the byproduct of Formula III.

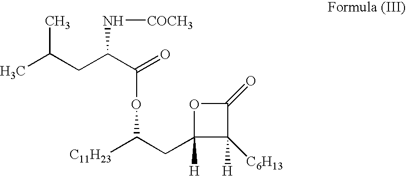

Embodiments of the process may include one or more of the following features. For example, the amino orlistat may be obtained as a solution directly from a reaction mixture in a process in which the amino orlistat is prepared. The amino orlistat may be obtained by deprotecting a protected amino orlistat.

The formic acid anhydride may be obtained by reacting formic acid with a coupling reagent in a suitable solvent. The coupling reagent may be one or both of N,N′-dicyclohexylcarbodimide and diisopropyl carbodiimide.

The solvent may be one or more of ethers, chlorinated hydrocarbons and mixtures thereof. The ether may be one or more of dioxane, tetrahydrofuran and mixtures thereof. The chlorinated hydrocarbon may be one or more of methylenedichloride, etlhylenedichloride and mixtures thereof.

The reaction of formic acid with the coupling reagent may be carried out at a temperature range of from about 0° C. to about −20° C. for a period of about thirty minutes to three hours.

The alkanoylation reaction may be performed in a solvent comprising one or more of ether, chlorinated hydrocarbon and mixtures thereof. The ether may be one or more of diethyl ether, methyl tert-butyl ether, dioxane, tetrahydrofuran and mixtures thereof. The chlorinated hydrocarbon may be one or more of methylene dichloride, ethylene dichloride and mixtures thereof. The alkanoylation reaction may be carried out at a temperature range of from about −10° C. to about 0° C.

The orlistat may be recrystallized with an aliphatic hydrocarbon that includes one or more of hexane, pentane, heptane, cyclohexane and mixtures thereof. The crystallisation may be performed at a temperature of from about 0° C. to about −10° C. for a period of thirty minutes to about twelve hours.

The details of one or more embodiments of the inventions are set forth in the description below. Other features, objects and advantages of the inventions will be apparent from the description and claims.

### DETAILED DESCRIPTION OF THE INVENTION

Amino orlistat may be obtained by methods known in the art including those described in U.S. Pat. No. 4,598,089; U.S. Pat. No. 4,983,746; U.S. Pat. No. 4,931,463 and WO 05/005403, which are incorporated herein by reference in their entirety. Amino orlistat may be obtained as a solution directly from a reaction mixture of the last step of a process in which it is prepared and used as such for the preparation of orlistat.

In general, amino orlistat may be obtained by deprotecting protected amino orlistat. Deprotection of protected amino orlistat may be carried out by alkali hydrolysis or basic hydrolysis based on the protecting group. Deprotection of protected amino orlistat may also be carried out by catalytic hydrogenation if the amino protecting group is a protecting group such as benzyloxycarbonyl or p-nitrobenzyloxycarbonyl, using palladium or platinum as metal catalyst.

The formic anhydride may be obtained by reacting formic acid with a coupling reagent in a suitable solvent.

Examples of coupling reagents include N,N′-dicyclohexylcarbodimide and diisopropyl carbodiimide. Examples of suitable solvents include ethers, such as dioxane and tetrahydrofuran, and chlorinated hydrocarbons, such as methylenedichloride and ethylenedichloride.

The addition of the coupling reagent may be carried out at a temperature range of from about 0° C. to about −20° C. The reaction of formic acid with the coupling reagent may be carried out at a temperature range of from about 0° C. to about −20° C. for a period of thirty minutes to three hours.

The alkanoylation of amino orlistat may be carried out in a suitable solvent, for example, ethers, such as dioxane and tetrahydrofuran, and chlorinated hydrocarbons, such as methylenedichloride and ethylenedichloride. The alkanoylation reaction of amino orlistat may be carried out at a temperature range of from about −10° C. to about 0° C.

The reaction mixture can be quenched by water, and extracted with a suitable solvent. The quenching of the reaction may be carried out at a temperature range of from about −5° C. to ambient temperature. The ambient temperature may be in the range of about 0° C. to about 30° C.

The solvent that may be used for the extraction of orlistat includes chlorinated hydrocarbons and esters. Examples of chlorinated hydrocarbons include methylenedichloride, ethylenedichloride and mixtures thereof. Examples of esters include ethyl acetate, isopropyl acetate and mixtures thereof.

Orlistat obtained may be recrystallized from a suitable solvent to obtain pure orlistat. The solvent that may be used for the crystallization of orlistat includes aliphatic hydrocarbons. Examples of aliphatic hydrocarbons include hexane, pentane, heptane, cyclohexane and mixtures thereof.

The crystallization may be performed at a temperature of from about 0° C. to about −10° C. for a period of about 30 minutes to about 12 hours.

The present invention demonstrates the use of a formic anhydride as an alkanoylating agent, which further aids the minimization of impurities in the product and results in a product of high purity substantially free of the byproduct of Formula III. The term “substantially pure” refers to the absence of byproduct of Formula III. The characteristic of being substantially free of the byproduct is further substantiated by experimental evidence, which is tabulated herein below:

| | HPLC Assay | Byproduct of | |
|---|---|---|---|
| Alkanoylating agent used | of Orlistat | Formula III | |
| for alkanoylation | obtained (% w/w) | (% w/w) | Yield % |
| Formic acid/ acetic acid | 99.5% | 0.699% | 82 |
| anhydride (Example 3) | | | |
| Formic acid anhydride | 99.2% | Not detected | 91 |
| (Example 4) | | | |

In the following section preferred embodiments are described by way of examples to illustrate the process. However, these are not intended in any way to limit the scope of the claims. Several variants of these examples would be evident to persons ordinarily skilled in the art.

### EXAMPLES

### Example 1

### Preparation of (S)-leucine (S)-1-[[(2S, 3S)-3-hexyl-4-oxo-2-oxetanyl]methyl dodecyl ester (amino orlistat)

A solution of the (S)-N-[(benzyloxy) carbonyl]leucine (S)-1-[[(2S, 3S)-3-hexyl-4-oxo-2-oxetanyl]methyl] dodecyl ester (25g, 0.0415 mol) in dichloromethane (100 mL) was hydrogenated in the presence of 10% palladium carbon (1.25 g, 50% moisture) at 25° C. to 30° C. under hydrogen atmosphere (2.5 to 3.0 Kg) for 1.5 hours. After completion of the reaction, the reaction mixture was filtered through a hyflo bed and the hyflo bed washed with dichloromethane (50 mL). The filtrate so obtained was used as such in the next step.

### Example 2

### Preparation of Formic Acid Anhydride

A mixture of formic acid (20 g, 0.434 mol) and dichloromethane (75 mL) was cooled to −10° C. A solution of N,N′-dicyclohexylcarbodimide (43 g, 0.208 mol) in dichloromethane (100 mL) was added slowly to the above mixture at −5° C. to −10° C. The reaction mixture was stirred at −5° C. to −10° C. for 2.5 hours. The reaction mixture was filtered through a hyflo bed at −10° C. and used as such for the allkanoylation reaction.

### Example 3

### Preparation of (S)-N-formyl leucine (S)-1-[[(2S, 3S)-3-hexyl-4-oxo-2 oxetanyl]methyl] dodecyl ester (orlistat)

To the solution of (S)-leucine (S)-1-[[(2S, 3S)-3-hexyl-4-oxo-2-oxetanyl]methyl] dodecyl ester (19.13g, 0.0415 mol) in dichloromethane (150 mL), formic acid/acetic anhydride reagent (obtained by mixing 25 g of formic acid in 13.75 g of acetic anhydride) was added slowly at −5° C. to −10° C. The reaction was monitored by TLC (ethyl acetate: hexane at 30: 70 v/v, I₂). After completion of the reaction, the reaction mixture was washed with water and sodium bicarbonate solution, and the dichloromethane was recovered completely. The residue was diluted with hexane and the resulting clear solution was treated with activated carbon, filtered and the solvent recovered under reduced pressure.

The orlistat residue was dissolved in hexane (175 ml) and the resulting hexane solution was cooled at 0 to −10° C. over a period of four to six hours. The temperature was maintained at 0 to −10° C. for a further six to seven hours and the resulting solid was filtered at 0 to −10° C. and dried at 30−35° C. under reduced pressure.

Yield: 15.75 gm (82%).

By-product of formula III: 0.699%.

Assay: 99.5 % by HPLC.

### Example 4

### Preparation of (S)-N-formyl leucine (S)-1-[[(2S, 3S)-3-hexyl-4-oxo-2 oxetanyl]methyl] dodecyl ester (Orlistat)

To the solution of (S)-leucine (S)-1-[[(2S, 3S)-3-hexyl-4-oxo-2-oxetanyl]methyl] dodecyl ester in dichloromethane (obtained from Example 1), formic acid anhydride solution (obtained from Example 2) was added slowly at −5° C. to −10° C. The reaction mixture was stirred for 1 hour at 0° C. to −5° C. After completion of the reaction, the reaction mixture was quenched with water (250 mL). The organic layer was separated and washed with sodium bicarbonate solution. The dichloromethane was recovered under reduced pressure. The residue was diluted with hexane and the resulting clear solution was treated with activated carbon. The mixture was filtered through a hyflo bed and the hyflo bed washed with hexane. Solvent was recovered under reduced pressure.

The orlistat residue was dissolved in hexane (175 ml) and the resulting hexane solution was cooled at 0 to −10° C. over a period of four to six hours. The temperature was maintained at 0 to −10° C. for a further six to seven hours and the resulting solid was filtered at 0 to −10° C. and dried at 30-35° C. under reduced pressure.

Yield: 17.5 gm (91%).

By-product of formula (III): Not detected

Assay: 99.2% by HPLC.

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim:

1. A process for the preparation of orlistat of Formula I, the process comprising 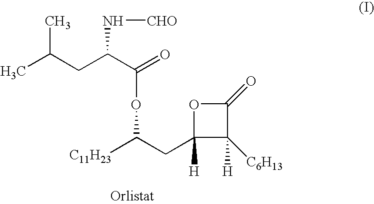
alkanoylating an amino orlistat of Formula II, 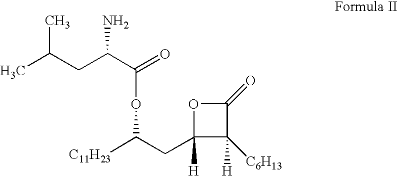
with formic acid anhydride to give orlistat of Formula I substantially free of the byproduct of Formula III.

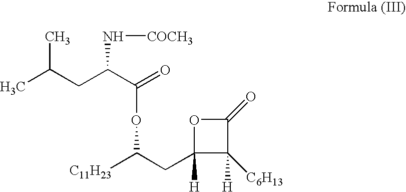

2. The process according to claim 1, wherein amino orlistat is obtained as a solution directly from a reaction mixture in a process in which the amino orlistat is prepared.

3. The process according to claim 1, wherein the amino orlistat is obtained by deprotecting a protected amino orlistat.

4. The process according to claim 1, wherein the formic acid anhydride is obtained by reacting formic acid with a coupling reagent in a suitable solvent.

5. The process according to claim 4, wherein the coupling reagent comprises one or both of N,N′-dicyclohexylcarbodimide and diisopropyl carbodiimide.

6. The process according to claim 4, wherein the solvent comprises one or more of ethers, chlorinated hydrocarbons and mixtures thereof.

7. The process according to claim 6, wherein the ether comprises one or more of dioxane, tetrahydrofuran and mixtures thereof.

8. The process according to claim 6, wherein the chlorinated hydrocarbon comprises one or more of methylenedichloride, ethylenedichloride and mixtures thereof.

9. The process according to claim 4, wherein the reaction of formic acid with the coupling reagent is carried out at a temperature range of from about 0° C. to about −20° C. for a period of about thirty minutes to three hours.

10. The process according to claim 1, wherein the alkanoylation reaction is performed in a solvent comprising one or more of ether, chlorinated hydrocarbon and mixtures thereof.

11. The process according to claim 10, wherein the ether comprises one or more of diethyl ether, methyl tert-butyl ether, dioxane, tetrahydrofuran and mixtures thereof.

12. The process according to claim 10, wherein the chlorinated hydrocarbon comprises one or more of methylene dichloride, ethylene dichloride and mixtures thereof.

13. The process according to claim 1, wherein the alkanoylation reaction is carried out at a temperature range of from about −10° C. to about 0° C.

14. The process according to claim 1, wherein the orlistat is recrystallized with an aliphatic hydrocarbon comprising one or more of hexane, pentane, heptane, cyclohexane and mixtures thereof.

15. The process according to claim 14, wherein the crystallisation is performed at a temperature of from about 0° C. to about −10° C. for a period of thirty minutes to about twelve hours.
